(19) United States
(12) Patent Application Publication
(10) Pub. No.: US 2013/0164755 A1
(43) Pub. Date: Jun. 27, 2013

(54) MICROFLUIDIC CHIP DEVICE FOR SELECTING A CELL APTAMER AND METHOD THEREOF

(71) Applicants: CHEN-HSUN WENG, Kaohsiung City (TW); GWO-BIN LEE, Hsinchu (TW); YUH-LING CHEN, Tainan (TW); SHU-CHU SHIESH, Tainan City (TW)

(72) Inventors: CHEN-HSUN WENG, Kaohsiung City (TW); GWO-BIN LEE, Hsinchu (TW); YUH-LING CHEN, Tainan (TW); SHU-CHU SHIESH, Tainan City (TW)

(73) Assignee: NATIONAL TSING HUA UNIVERSITY, Hsinchu (TW)

(21) Appl. No.: 13/462,350

(22) Filed: May 2, 2012

(30) Foreign Application Priority Data
Dec. 21, 2011 (TW) 100147640

(51) Int. Cl. C12Q 1/68; C12M 1/40

(52) U.S. Cl. 435/6.12; 435/287.2

(57) ABSTRACT

The present invention provides a microfluidic chip device for selecting a cell aptamer. The microfluidic chip device comprising a plurality of storage reservoirs; a fluid control unit; a reaction tank; and a PCR reaction tank, wherein each storage reservoir interconnects with the fluid control unit, and via a corresponding pumping/mixing element, the sample and the reagent are mixed and then transported into each storage reservoir. The present invention further provides a method for selecting a cell aptamer.

### FIELD OF THE INVENTION

The present invention relates to a microfluidic chip device for selecting a cell aptamer and method thereof.

### Description of Prior Art

Screening tumor cell-specific molecular markers is important in tumor diagnosis and target therapy. Recently, the systematic evolution of ligands by exponential enrichment (SELEX) technology has been developed to screen specific ligands, usually referred as aptamers by performing reiterated cycles of enrichment and amplification of single-strain DNA (ssDNA). The characteristics of the screened aptamers have potential applications, such as sample purification, target validation, drug development, diagnostics, and even therapy.

The conventional device shown in Taiwan patent application number 201040523 provides a microfluidic chip for proceeding SELEX. The device is characterized by decreasing sample amount due to liquid flow and container change, and then achieving the anticipative reaction by using few samples; however, the conventional device discloses the system which merely comprises a reaction tank, that is, all reactions are carried out in the reaction tank. Thus, the remaining agents of pre-reaction could affect the subsequent reaction.

In the past, in the traditional SELEX, performing the purification step was in the eppendorf; however, during washing, due to that the sample could be easily remained in the bottom of the eppendorf, the subsequent reaction will be affected. Furthermore, the trivial artificial operating procedures consume much more time, when comparing with the present invention as shown in Table 1. For example, in the conventional technique, it is difficult to extract a trace of sample and the operation of a large machine needs to spend much more time. The above drawbacks cause waste on the potential cost and resources.

TABLE 1
the required time of SELEX operation process and step

| | Mins | | | | | | |
|---|---|---|---|---|---|---|---|
| | | Culture | | | Culture | | |
| method | Denaturation | target cell | Cleaning | Cell lysis | control cell | PCR | Total time |
| Traditional | 5 mins | 60 mins | 15 mins | 5 mins | 60 mins | 45 mins | 3 hours |
| method | | | | | | | 10 mins |
| The chip device | 5 mins | 20 mins | 5 mins | 5 mins | 20 mins | 45 mins | 1 hour |
| of the present | | | | | | | 40 mins |
| invention | | | | | | | |

In view of the drawbacks of those prior art devices, i.e. not effective to obtain the aptamers. It is important to develop a microfluidic chip device with a high affinity and specificity to obtain the aptamers of subject matter.

### BRIEF DESCRIPTION OF THE DRAWINGS

FIG. 1 is a schematic view showing the microfluidic chip device of the present invention.

FIG. 2 is a schematic view showing the controlling temperature unit of the present invention.

FIG. 3 shows the operation of the microfluidic chip device for selecting samples (target cell or control cell).

FIG. 4 is electrophoresis images showing the samples are selected via SELEX process in the microfluidic chip device. (Lane 1 shows the positive control; Lane 2 shows the first washing; Lane 3 shows the second washing; Lane 4 shows the third washing; Lane 5 shows PCR product is amplified through 5 rounds by the microfluidic chip device of the present invention; Lane 6 shows the PCR product is amplified through 6 rounds by the microfluidic chip device system of the present invention).

FIG. 5 is electrophoresis images showing the target cells are selected by SELEX reaction repeatedly in the microfluidic chip device. (Lane A shows that the target cells are washed by cleaning solution; Lane B shows that the target cells dissolve in the buffer; Lane C shows that only single-strain deoxyribonucleic acid (ssDNA); Lane D shows that the target cells are performed SELEX reaction through 14 rounds; Lane E shows that the target cells are performed SELEX reaction through 15 rounds; Lane F shows that the target cells are performed SELEX reaction through 16 rounds.

FIG. 6 is electrophoresis images showing the lung cancer cell aptamers are selected by the microfluidic chip device of the present invention. (Lane A shows positive reaction; Lane B shows only lung cancer cells (H1650) mix the selected aptamer; Lane C shows only ovarian cancer cells (BG1) mix the selected aptamer).

### SUMMARY OF THE INVENTION

The present invention provides a microfluidic chip device comprising: (a) a plurality of storage reservoirs; (b) a fluid control unit, including a plurality of channels, a plurality of pumping/mixing elements and/or a plurality of valves, for controlling at least a sample and/or at least a reagent to be transported in the plurality of storage reservoirs, wherein the sample is a plurality of nucleic acids to be selected, a plurality of target cells to be selected or a plurality of control cells to be selected; (c) a reaction tank connected to the fluid control unit, for mixing or purifying the sample and/or the reagent; and (d) a PCR reaction tank connected to a PCR regent storage reservoir which provides a PCR regent, the PCR reaction tank is used for performing a PCR reaction of the nucleic acid to be selected so as to obtain a cell aptamer to be selected, wherein each storage reservoir interconnects with the fluid control unit, and via a corresponding pumping/mixing element, the sample and the reagent are mixed and then transported into each storage reservoir.

The present invention further provides a method for selecting a cell aptamer, comprising steps of: (a) providing a microfluidic chip device of the present invention; (b) providing a plurality of nucleic acids to be selected and a plurality of target cells to be selected, and then mixing the nucleic acids with the target cells to be selected; (c) purifying and rinsing the nucleic acids and the target cells to be selected in step (b); (d) lysing the target cells to produce a substrate; (e) mixing the substrate and a plurality of control cells to obtain a substance; (f) purifying the substance and the control cells in step (e) to obtain a purified substrate; and (g) performing polymerase chain reaction (PCR) from the purified substrate in step (f) to obtain the cell aptamer to be selected.

### DETAILED DESCRIPTION OF THE INVENTION

Unless defined otherwise, all technical and scientific terms used herein have the same meaning as commonly understood to one of ordinary skill in the art to which this invention pertains. In this application, certain terms are used, which shall have the meanings as set in the specification. It must be noted that as used herein and in the appended claims, the singular forms “a,” “an,” and “the” include plural reference unless the context clearly dictates otherwise.

The term “nucleic acid molecular” used herein, unless otherwise indicated, means a biological molecule that locates in the nucleus, and acts as carrier and transmission of heredity message of organism, further comprises a single strand deoxyribonucleotide and a double strand deoxyribonucleotide.

The term “non-nucleic acid molecular” used herein, unless otherwise indicated, means an amino acid, a protein, a drug, a small organic molecule or an aptamer.

The term “aptamer” used herein, unless otherwise indicated, means a DNA or RNA sequence which could be selected from nucleic acid molecules library by SELEX.

The term “select nucleic acid” used herein, unless otherwise indicated, means that selecting the aptamer with high specificity and affinity from the target cell or control cell.

In view of the drawbacks of the traditional SELEX, the present invention discloses a microfluidic chip device for selecting a cell aptamer and a using method thereof, wherein the present invention provides a microfluidic chip device which comprises: (a) a plurality of storage reservoirs; (b) a fluid control unit, including a plurality of channels, a plurality of pumping/mixing elements and/or a plurality of valves, for controlling at least a sample and/or at least a reagent to be transported in the plurality of storage reservoirs, wherein the sample is a plurality of nucleic acids to be selected, a plurality of target cells to be selected or a plurality of control cells to be selected; (c) a reaction tank connected to the fluid control unit, for mixing or purifying the sample and/or the reagent; and (d) a PCR reaction tank connected to a PCR regent storage reservoir which provides a PCR regent, the PCR reaction tank is used for performing a PCR reaction of the nucleic acids to be selected to obtain a cell aptamer so as to be selected, wherein each storage reservoir interconnects with the fluid control unit, and via a corresponding pumping/mixing element, the sample and the reagent are mixed and then transported into each storage reservoir.

In some embodiments of the present invention, the storage reservoir further comprises a nucleic acid storage reservoir to be selected, a target cell storage reservoir, a control cell storage reservoir, a cleaning solution storage reservoir, a waste liquid storage reservoir, a buffer storage reservoir, a lysising cell storage reservoir and/or the PCR regent storage reservoir.

In some embodiments of the present invention, the cleaning solution storage reservoir stores a cleaning solution that comprising Dulbecco's phosphate-buffered saline, glucose, or/and MgCl₂, and the buffer comprises Dulbecco's phosphate-buffered saline, glucose, bovine serum albumin (BSA), transfer RNA (tRNA) or/and MgCl₂.

In some embodiments of the present invention, each of the plurality of pumping/mixing elements and/or of the plurality of valves of the fluid control unit connects one end of an electromagnetic valve, the other end of the electromagnetic valve connects a control circuit, and via a software to control a switch of the electromagnetic valve.

In some embodiments of the present invention, the microfluidic chip device further comprises a temperature controlling unit for modulating the temperature variation of the microfluidic chip device, and the temperature controlling unit further comprises a heating region and a cooling region. Furthermore, the heating region is located at the nucleic acid storage reservoir, the lysising cell storage reservoir and/or the PCR reservoir, and the cooling region is located at the target cell storage reservoir, the control cell storage reservoir, the buffer storage reservoir and/or the PCR regent storage reservoir.

In some embodiments of the present invention, a bottom side and/or a lateral side of the reaction tank is installed with a unit for generating a magnetic field, and the unit for generating a magnetic field is a microcoil array, a ferrite magnet, an NdFeB magnet or a combination of the above.

In some embodiments of the present invention, the sample is a cancer cell, a stem cell and/or a normal cell.

The present invention further provides a method for selecting a cell aptamer, comprising steps of: (a) providing a microfluidic chip device of the present invention; (b) providing a plurality of nucleic acids to be selected and a plurality of target cells to be selected, and then mixing the nucleic acids with the target cells to be selected; (c) purifying and rinsing the nucleic acids and the target cells to be selected in step (b); (d) lysing the target cells to produce a substrate; (e) mixing the substrate and a plurality of control cells to obtain a substance; (f) purifying the substance and the control cells in step (e) to obtain a purified substrate; and (g) performing polymerase chain reaction (PCR) from the purified substrate in step (f) to obtain the cell aptamer to be selected.

In some embodiments of the present invention, the step further comprises opening a cool actuating unit before step (b) to reserve at least a sample and/or at least a reagent.

In some embodiments of the present invention, the sample is a nucleic acid, a target cell or a control cell, wherein the target cell or the control cell is linked to a plurality of magnetic beads.

In some embodiments of the present invention, the target cell or the control cell is a cancer cell, a stem cell and/or a normal cell.

In some embodiments of the present invention, the steps (b)-(g) of the operation steps are performed in the microfluidic chip device.

### EXAMPLES

The examples below are non-limiting and are merely representative of various aspects and features of the present invention.

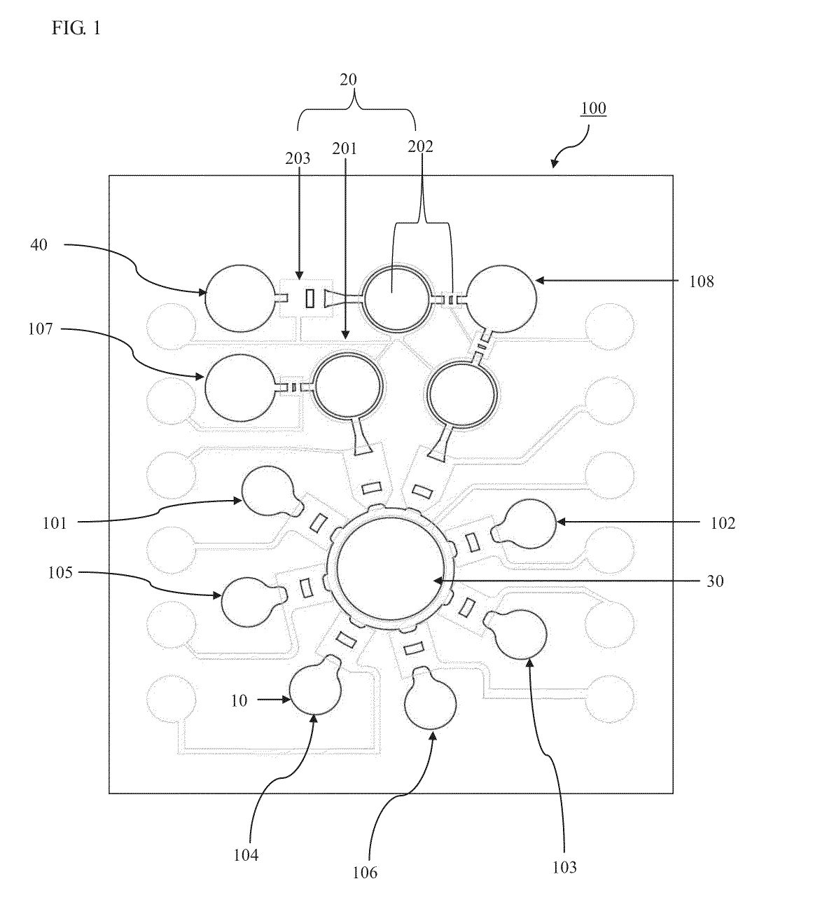

Referring to FIG. 1, the present invention provides a microfluidic chip device 100 comprising a plurality of storage reservoirs 10; a fluid control unit 20, including a plurality of channels 201, a plurality of pumping/mixing elements 202 and/or a plurality of valves 203, for controlling at least a sample and/or at least a reagent to be transported in the plurality of storage reservoirs 10, wherein the sample is a plurality of nucleic acids to be selected, a plurality of target cells to be selected or a plurality of control cells to be selected; a reaction tank 30 connected to the fluid control unit 20 for mixing or purifying the sample and/or the reagent; and a PCR reaction tank 40 connected to a PCR regent storage reservoir 108 which provides a PCR regent, the PCR reaction tank 40 is used for performing a PCR reaction of the nucleic acids to be selected to obtain a cell aptamer to be selected, wherein each storage reservoir 10 interconnects with the fluid control unit 20, and via a corresponding pumping/mixing element 202, the sample and the reagent are mixed and then transported into each storage reservoir 10.

The plurality of storage reservoirs 10 of the present invention further comprise a nucleic acid storage reservoir 101, a target cell storage reservoir 102, a control cell storage reservoir 103, a cleaning solution storage reservoir 104, a waste liquid storage reservoir 105, a buffer storage reservoir 106, a lysising cell storage reservoir 107 and/or the PCR regent storage reservoir 108.

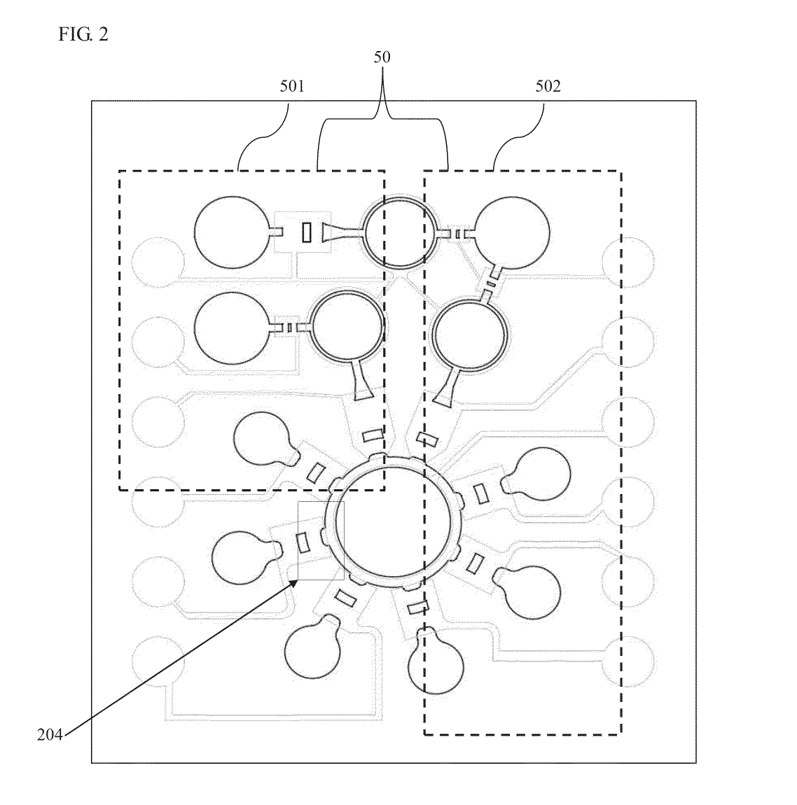

Referring to FIG. 2, the microfluidic chip device 100 further comprises a temperature controlling unit 50 for modulating the temperature variation of the microfluidic chip device 100 of the present invention, in which the temperature controlling unit 50 further comprises a heating region 501 and a cooling region 502.

The examples below are non-limiting and are merely representative of various aspects and features of the present invention.

The present invention uses the microfluidic chip device to select a cancer cell aptamer. Moreover, the detailed components, implementing steps and method of the microfluidic chip device are illustrated. FIGS. 1, 2 and 3 are described in the examples.

### Example 1

### Preliminary Process of the Target Cell

The target cell of the present invention was lung cancer cell (H1650), and the control cell was ovarian cancer cell (BG1).

The antibodies had capability of grabbing cancer cell after the beads were modified antibodies. The binding reaction in above preprocess was at a room temperature, in which the cancer cell was bind beads, and then injected into the microfluidic chip device.

### Example 2

### The Processes for Performing the Microfluidic Chip Device of the Present Invention

Preprocessing: the reagents and samples (the target cell 601 or the control cell was injected into a corresponding storage reservoir 10.

Step 1: The temperature was raised to 95° C. on the heating region 501, and the random deoxyribonucleic acids were denatured into the random single strand deoxyribonucleic acids 602.

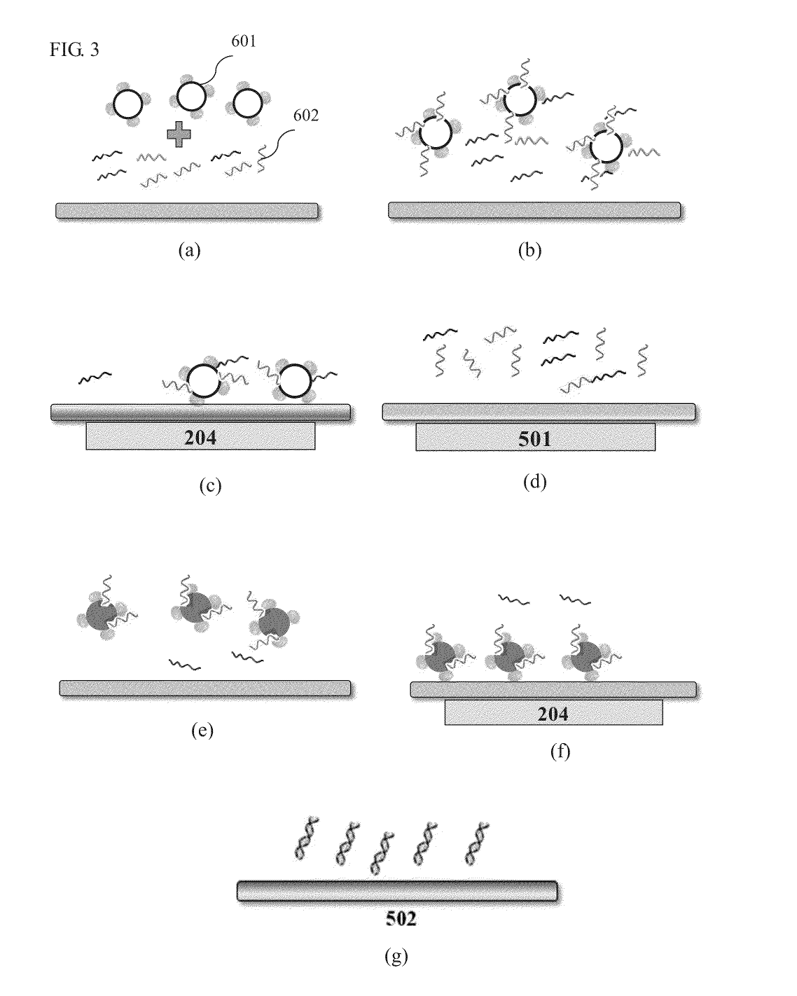

Step 2: The target cells with magnetic beads (cancer cells) 601 and the random single strand deoxyribonucleic acids 602 were injected the reaction tank 30, and the pumping/mixing elements 202 were opened to mix the target cells with magnetic beads (sample) 601 and the random single strand deoxyribonucleic acids 602 for 15˜25 minutes. The random single strand deoxyribonucleic acids 602 and the target cells with magnetic beads (sample) 601 reached to mix effectively, then it would be predicted that some single strand deoxyribonucleic acids 602 could be attached the target cells 601 (shown in FIG. 3(b)).

Step 3: It started to produce a magnetic field 204 on the bottom side or lateral side of the reaction tank 30, and the target cells with magnetic beads were adsorbed on the bottom of the microfluidic chip device (shown in FIG. 3(c)).

Step 4: The cleaning solution in the cleaning solution storage reservoir 104 was transmitted into the reaction tank 30, and the non-binding random single strand deoxyribonucleic acids and the cleaning solution were transmitted into the waste liquid storage reservoir 105 by the pumping/mixing elements 202. Under the control of the electromagnetic valve at each time, the non-binding random single strand deoxyribonucleic acid could be cleaned by repeating three times in the present step.

Step 5: After completing the clean step, and the pumping/mixing elements 202 and means for producing the magnetic field 204 were closed, under the control of the electromagnetic valve, the buffer which used for binding function was transmitted into the reaction tank 30, and then the cancer cells which bind random single strand deoxyribonucleic acids were dissolved in buffer and the cancer cells transmitted into lysising cell storage reservoir 107.

Step 6: Opening the heating region 501 and heating to 50° C., the cancer cells were lysed and the single strand deoxyribonucleic acids were dissociated, wherein this step proceeded in lysising cell storage reservoir 107 (shown in FIG. 3(d)).

Step 7: The single strand deoxyribonucleic acids which were stored in lysising cell storage reservoir 107 in Step 6 were further transmitted into the reaction tank 30.

Step 8: The control cells with magnetic beads 603 were transmitted into the reaction tank 30, then opening the pump/mixer 202 to mix the single strand deoxyribonucleic acids (Step 7) and the control cells with magnetic beads 603 for 15˜25 minutes. Predicting some single strand deoxyribonucleic acids could also attach the control cells with magnetic beads 603, and some free single strand deoxyribonucleic acids were in the solution, that is, the free single strand deoxyribonucleic acids had great specificity to target cells (shown in FIG. 3(e)).

Step 9: he free single strand deoxyribonucleic acids (about 2 μl) in steps 8 were transmitted the PCR reservoir storage reservoir 108, and the free single strand deoxyribonucleic acids and PCR reagent mixed uniformly.

Step 10: The samples in step 9 and whole reaction reagent (comprising PCR reagent) were transmitted into the PCR reservoir 40, and performing polymerase chain reaction. In addition, a mineral oil was injected into the PCR reservoir 40 to prevent samples and reagent evaporating (shown in FIG. 3(f)).

Step 11: The polymerase chain reaction was proceed in PCR reservoir 40 via the temperature controlling unit 50 which produced accurate temperature, and the single strand deoxyribonucleic acids by extraction method of the magnetic beads were amplified into the double stand deoxyribonucleic acids (shown in FIG. 3(g)).

Step 12: The PCR products were taken out from the PCR reservoir 40 (about 3 μl), while repeating from Step 1 to Step 11, and new cancer cells (target cells and control cells) and reaction reagents were added in suitable step. A round was from Step 1 to Step 12, and rounds were repeated to obtain the cell aptamer to be selected.

### Example 3

### The Results of the Target Cells were Reacted in the Microfluidic Chip Device

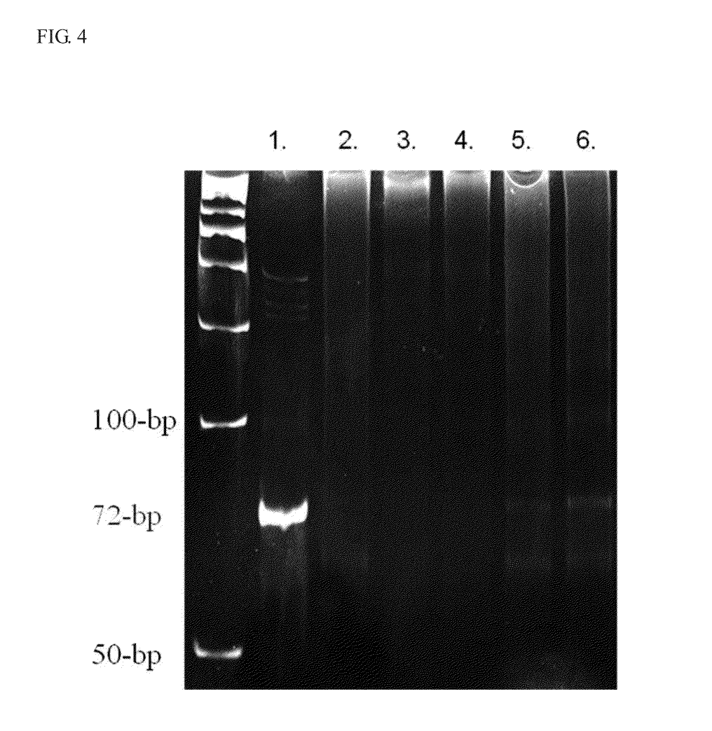

After the operation steps of Example 2, the PCR products were took out, and were analyzed by electrophoretic, in which the results were referred to FIGS. 4-6.

FIG. 4 showed electrophoresis images. The random single strand deoxyribonucleic acids and target cells were cleaned three times repeatedly via the cleaning solution in Step 4, and took out the sample (random single strand deoxyribonucleic acids and target cells) in the waste liquid storage reservoir, then further examined cleaning condition of non-bound single strand deoxyribonucleic acids.

FIG. 4 showed after cleaned three times repeatedly, the waste solution of each time was amplified via PCR, and the results showed there was not any single strand deoxyribonucleic acids in the reaction tank. As FIG. 4 showed, Lane 1 was the positive control; Lane 2 was the first washing; Lane 3 was the second washing; Lane 4 was the third washing; Lane 5 was PCR product was amplified through 5 rounds by the microfluidic chip device of the present invention; Lane 6 was the PCR product was amplified through 6 rounds by the microfluidic chip device system of the present invention.

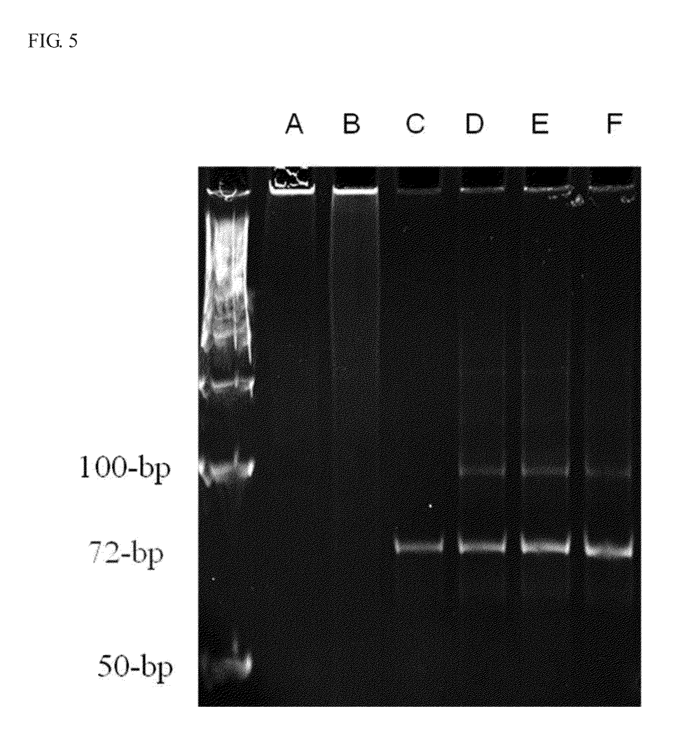

FIG. 5 showed the samples were performed by SELEX reaction after 14, 15 and 16 rounds, respectively. The results were showed in Lane D, Lane E and Lane F, respectively. In addition, FIG. 5 showed when samples were performed many times by SEXLEX reaction. With reaction rounds increased, the signal could be enhanced. Thus, certainly the aptamer was selected by the microfluidic chip device from target cell.

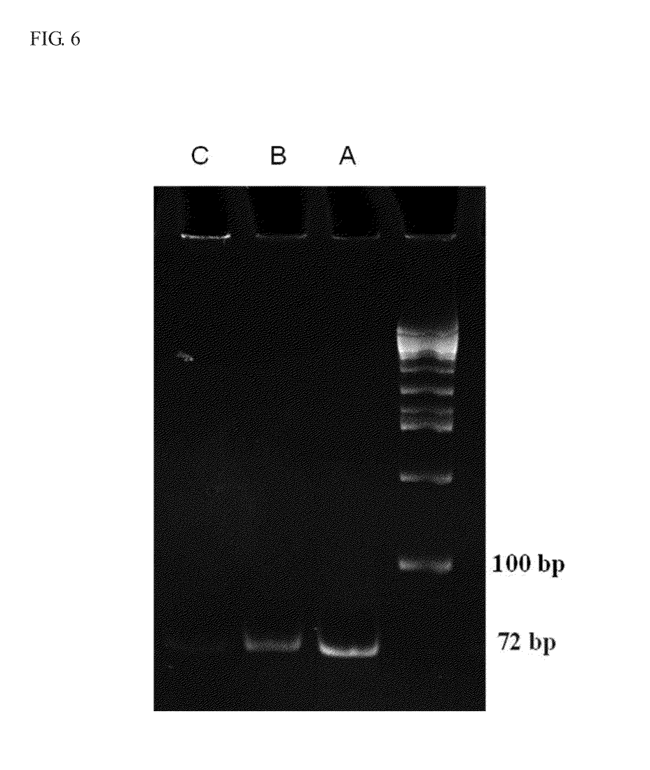

FIG. 6 showed electrophoresis images. Lane A was regarded as positive reaction that the aptamer was selected by the microfluidic chip device of the present invention. Lane B meant that only lung cancer cells (H1650) mixed the selected aptamer. Lane C meant that only mixed ovarian cancer cell (BG1) mixed the selected aptamer. FIG. 6 showed that signal B was stronger than signal C. Thus, synthesized above results, during micofluidic chip device of the present invention process, the cell-specific DNA sequences (with length of 72 bps) of the lung cancer cell (target cell) could be successfully separated and enriched. The selected aptamer had affinity and specificity for lung cancer cell.

The automatic and rapid operating platform of the microfluidic chip device of the present invention could replace the traditional SELEX procedure. Moreover, the present invention wasted very low cost and consumed fewer sample to obtain the selective purpose. On the other hand, performing the magnetic-bead operation technology in the invention could largely decrease operation time and inconveniency as compared with the traditional technology, while reducing contaminated risk of the sample.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. Moreover, due to biological functional equivalency considerations, changes can be made in protein structure without affecting the biological action in kind or amount. All such modifications are intended to be included within the scope of the appended claims.

What is claimed is:

1. A microfluidic chip device comprising:
(a) a plurality of storage reservoirs;
(b) a fluid control unit, including a plurality of channels, a plurality of pumping/mixing elements and/or a plurality of valves, for controlling at least a sample and/or at least a reagent to be transported in the plurality of storage reservoirs, wherein the sample is a plurality of nucleic acids to be selected, a plurality of target cells to be selected or a plurality of control cells to be selected;
(c) a reaction tank connected to the fluid control unit, for mixing or purifying the sample and/or the reagent; and
(d) a PCR reaction tank connected to a PCR regent storage reservoir which provides a PCR regent, the PCR reaction tank is used for performing a PCR reaction of the nucleic acid so as to be selected to obtain a cell aptamer to be selected,
wherein each storage reservoir interconnects with the fluid control unit, and via a corresponding pumping/mixing element, the sample and the reagent are mixed and then transported into each storage reservoir.

2. The microfluidic chip device of claim 1, wherein the storage reservoir further comprises a nucleic acid storage reservoir, a target cell storage reservoir, a control cell storage reservoir to be selected, a cleaning solution storage reservoir, a waste liquid storage reservoir, a buffer storage reservoir, a lysising cell storage reservoir and/or the PCR regent storage reservoir.

3. The microfluidic chip device of claim 1, wherein each of the plurality of pumping/mixing elements and/or of the plurality of valves connects one end of an electromagnetic valve, and the other end of the electromagnetic valve connects a control circuit for controlling the electromagnetic valve.

4. The microfluidic chip device of claim 1, further comprising a temperature controlling unit in the microfluidic chip device.

5. The microfluidic chip device of claim 4, wherein the temperature controlling unit comprises a heating region and a cooling region.

6. The microfluidic chip device of claim 5, wherein the heating region is located at the nucleic acid storage reservoir, the lysising cell storage reservoir and/or the PCR reservoir.

7. The microfluidic chip device of claim 5, wherein the cooling region is located at the target cell storage reservoir, the control cell storage reservoir, the buffer storage reservoir and/or the PCR regent storage reservoir.

8. The microfluidic chip device of claim 1, wherein a bottom side and/or a lateral side of the reaction tank is installed with a unit for generating a magnetic field.

9. The microfluidic chip device of claim 1, the sample is a cancer cell, a stem cell and/or a normal cell.

10. A method for selecting a cell aptamer, comprising steps of :
(a) providing a microfluidic chip device as claimed in claim 1;
(b) providing a plurality of nucleic acids to be selected and a plurality of target cells to be selected, and then mixing the nucleic acids with the target cells to be selected;
(c) purifying and rinsing the nucleic acids and the target cells to be selected in step (b);
(d) lysing the target cells to produce a substrate;
(e) mixing the substrate and a plurality of control cells to obtain a substance;
(f) purifying the substance and the control cells in step (e) to obtain a purified substrate; and
(g) performing polymerase chain reaction (PCR) from the purified substrate in step (f) to obtain the cell aptamer to be selected.

11. The method of claim 10, wherein the steps further comprising opening a cool actuating unit before step (b) to reserve at least a sample and/or at least a reagent.

12. The method of claim 11, wherein the sample is a nucleic acid, a target cell or a control cell.

13. The method of claim 10, wherein the target cell or the control cell is linked to a plurality of magnetic beads.

14. The method of claim 10, wherein the target cell or the control cell is a cancer cell, a stem cell and/or a normal cell.

15. The method of claim 10, wherein the steps (b)-(g) are proceeded in the microfluidic chip device.
